(19) United States
(12) Patent Application Publication
(10) Pub. No.: US 2013/0066217 A1
(43) Pub. Date: Mar. 14, 2013

(54) SPHYGMOMANOMETER

(71) Applicant: Yasuo MATSUNAMI, Chiba (JP)

(72) Inventor: Yasuo MATSUNAMI, Chiba (JP)

(21) Appl. No.: 13/591,417

(22) Filed: Aug. 22, 2012

(30) Foreign Application Priority Data
Sep. 9, 2011 (JP) 2011-196940

(51) Int. Cl. A61B 5/022

(52) U.S. Cl. 600/499

(57) ABSTRACT

A main body of a sphygmomanometer includes an air duct having an end connected to a tube extending from the cuff and another end connected to the inflation bulb. The main body also includes a pressure sensor that measures a pressure in the air duct, a display unit, a controller that controls display operation of the display unit in accordance with a measurement obtained using the pressure sensor, and a determination button. In the sphygmomanometer, the cuff, the tube, the air duct and the inflation bulb communicate with one another so as to have a uniform inner pressure, and the controller causes the pressure value measured when the determination button is pressed to be continuously displayed in the display unit.

### BACKGROUND OF THE INVENTION

1. Field of the Invention

The present invention relates to simple electronic sphygmomanometers.

2. Description of the Related Art

Existing manual sphygmomanometers are roughly classified into mercury sphygmomanometers and aneroid sphygmomanometers.

The mercury sphygmomanometers are considered to be most reliable. As illustrated in FIG. 7, the mercury sphygmomanometers have the cuff 3 (bag-like belt to be wrapped around an upper arm) that has two tubes 4: one of the tubes 4 is connected to the mercury sphygmomanometer 5: another one of the tubes 4 is connected to an inflation bulb 2.

As illustrated in FIG. 8, the aneroid sphygmomanometers have the cuff 3 (bag-like belt to be wrapped around an upper arm) that has two tubes 4: one end of the tubes 4 is connected to an aneroid gauge 6: another end of the tubes 4 is connected to an inflation bulb 2. Although the measurement accuracy of the aneroid sphygmomanometer is lower than that of the mercury sphygmomanometer, the aneroid sphygmomanometer is handy to carry about.

Such manual sphygmomanometers are generally operated as follows: Initially, the cuff is wrapped around an upper arm leaving a space of one or two fingers slipped in between the cuff and the upper arm. A diaphragm (head) of stethoscope is applied to the center of a flexor of the elbow joint (artery part). Air is pumped into the cuff using the inflation bulb so as to set the cuff pressure to be greater than an expected systolic blood pressure. Next, the valve of the inflation bulb is loosened so as to gradually decrease the cuff pressure while sounds are listened for from the stethoscope. When Korotkoff sounds are heard, the cuff pressure is memorized as the systolic blood pressure. The cuff pressure is further gradually decreased and when the Korotkoff sounds became inaudible, the cuff pressure is memorized as the diastolic blood pressure. It is suggested that the above-described operation is performed at least twice to take the average of measured values.

Nowadays, automatic sphygmomanometers are widely used. Such sphygmomanometers are, for example, disclosed in Japanese Unexamined Patent Application Publications No. 5-207981 and No. 5-344956. Automatic sphygmomanometers automatically increase and decrease the cuff pressure using motors and detect Korotkoff sounds using acoustic sensors incorporated in the cuffs, thereby displaying systolic and diastolic blood pressures. Computer control is employed in order to drive the motors, analyze Korotkoff sounds, control display, and so forth.

### SUMMARY OF THE INVENTION

In the case where blood pressures are manually measured, the manometer is read while sounds from the stethoscope are listened for. This may lead to a delay in reading the manometer, and accordingly, to incorrect measurement. Furthermore, since blood pressures having been read are memorized, the read values may be incorrectly memorized.

Although mercury sphygmomanometers are regarded as most reliable sphygmomanometers, use of mercury is not desirable in terms of environmental protection.

In blood pressure measurement using automatic sphygmomanometers, Korotkoff sounds are identified in a manner standardized by a computer program. As a result, the presence or absence of Korotkoff sounds is not correctly determined due to causes such as the individual difference in vascular murmur and accordingly there is a possibility of occurrence of large measurement errors. Thus, blood pressure measured using automatic sphygmomanometers are regarded only as reference values.

An object of the present invention is to facilitate blood pressure measurement so as to decrease the work performed by the operator while maintaining advantages of manual blood pressure measurement, and is to reduce delays in reading a manometer and is to prevent incorrect memories of read blood pressures, thereby improving the accuracy of blood pressure measurement.

According to an aspect of the present invention, a sphygmomanometer includes a main body, a cuff, and an inflation bulb. In the sphygmomanometer, the main body includes an air duct having an end connected to a tube extending from the cuff and another end connected to the inflation bulb. The main body also includes a pressure sensor that measures a pressure in the air duct, a display unit, a controller that controls display operation of the display unit in accordance with a measurement obtained using the pressure sensor, and a determination button. In the sphygmomanometer, the cuff, the tube, the air duct and the inflation bulb communicate with one another so as to have a substantially uniform inner pressure, and the controller causes the display unit to continuously display the pressure in the air duct measured when the determination button is pressed.

The display unit may include a systolic blood pressure display and a diastolic blood pressure display. In this case, the pressure in the air duct measured when the determination button is pressed for the first time during a process of decreasing the pressure in the air duct is continuously displayed as the systolic blood pressure. Also in this case, the pressure in the air duct measured when the determination button is pressed for the second time is continuously displayed as the diastolic blood pressure. Also in this case, operations of the display unit to display the systolic blood pressure and the diastolic blood pressure are controlled by the controller.

The controller may cause the systolic blood pressure display to be reset when the pressure in the air duct is increased due to repressurization after the systolic blood pressure has been displayed. In this case, the pressure in the air duct measured when the determination button is pressed after the systolic blood pressure display has been reset is continuously displayed as the systolic blood pressure.

The controller may cause the diastolic blood pressure display to be reset when the pressure in the air duct is increased up to be equal to or smaller than the pressure in the systolic blood pressure display due to repressurization after the diastolic blood pressure has been displayed. In this case, the pressure in the air duct measured when the determination button is pressed after the diastolic blood pressure display has been reset is continuously displayed as the diastolic blood pressure.

The controller may cause the systolic blood pressure display and the diastolic blood pressure display to be reset when the pressure in the air duct is increased up to be greater than the pressure in the systolic blood pressure display due to repressurization after the diastolic blood pressure has been displayed. In this case, the pressure in the air duct measured when the determination button is pressed after the systolic blood pressure display and the diastolic blood pressure display have been reset is continuously displayed as the systolic blood pressure.

The display unit may further include a cuff pressure display. In this case, the controller causes the cuff pressure display to display the real time pressure in the air duct.

In order to use the sphygmomanometer according to the present invention, the cuff is wrapped around an upper arm of the subject. A diaphragm (head) of stethoscope is applied to the center of a flexor of the elbow joint (artery part). Air is pumped into the cuff using the inflation bulb so as to set the cuff pressure to be greater than an expected systolic blood pressure. Next, the valve of the inflation bulb is loosened so as to gradually decrease the cuff pressure while sounds from the stethoscope are listened for. When Korotkoff sounds are heard, the determination button is pressed. Thus, the systolic blood pressure is continuously displayed in the display unit. The cuff pressure is gradually decreased further and the determination button is pressed when Korotkoff sounds become inaudible. Thus, the diastolic blood pressure is continuously displayed in the display unit. When measurement of the blood pressure is completed, the display unit displays the systolic blood pressure and the diastolic blood pressure.

### Advantages

With the sphygmomanometer according to the present invention, the operator only needs to listen for sounds from the stethoscope and press the determination button. Thus, there is no need to read the manometer, and it is possible to concentrate on listening for and hearing the sounds from the stethoscope. This reduces the possibility of delay in reading a blood pressure. In case the determination button is pressed at a wrong timing, measurement can be performed again by repressurization. Since the measured values are continuously displayed in the display unit, mistakes caused by incorrect memories of the measured values are reduced.

### BRIEF DESCRIPTION OF DRAWINGS

FIG. 1 is an outline view of a sphygmomanometer according to an embodiment.

FIG. 2 illustrates a longitudinal sectional view of a main body and a side view of an inflation bulb.

FIG. 3 is a plan view of the main body and the inflation bulb.

FIG. 4 is a plan view of a display unit.

FIG. 5 is a block diagram of the main body.

FIG. 6 is a chart illustrating an example of blood pressure measurement.

FIG. 7 is an outline view of a mercury sphygmomanometer.

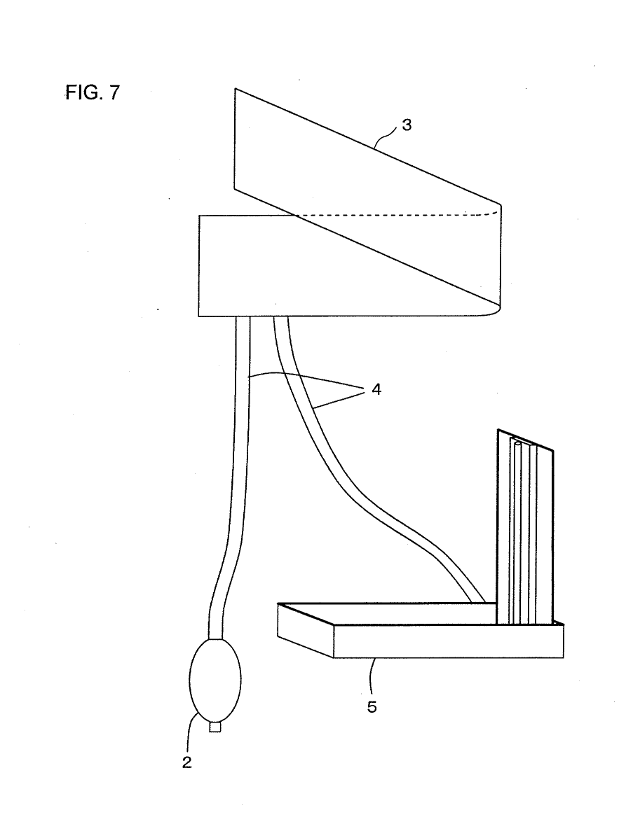

FIG. 8 is an outline view of an aneroid sphygmomanometer.

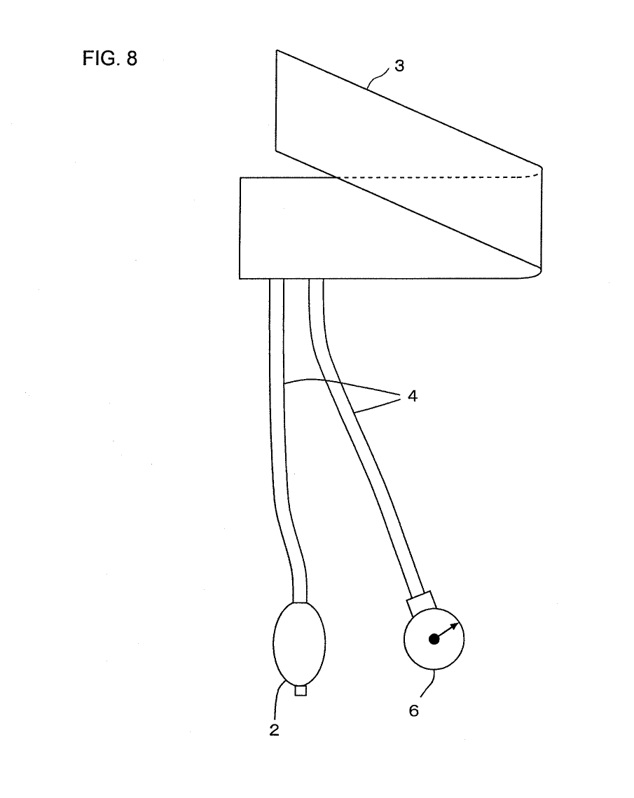

### DESCRIPTION OF THE PREFERRED EMBODIMENTS

The present invention will be described in detail below with reference to the drawings that illustrate an embodiment.

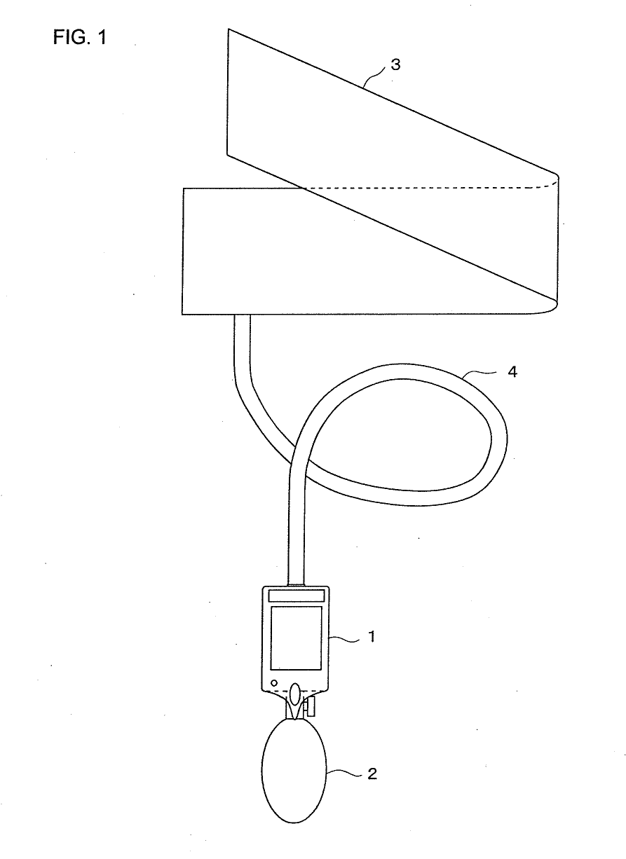

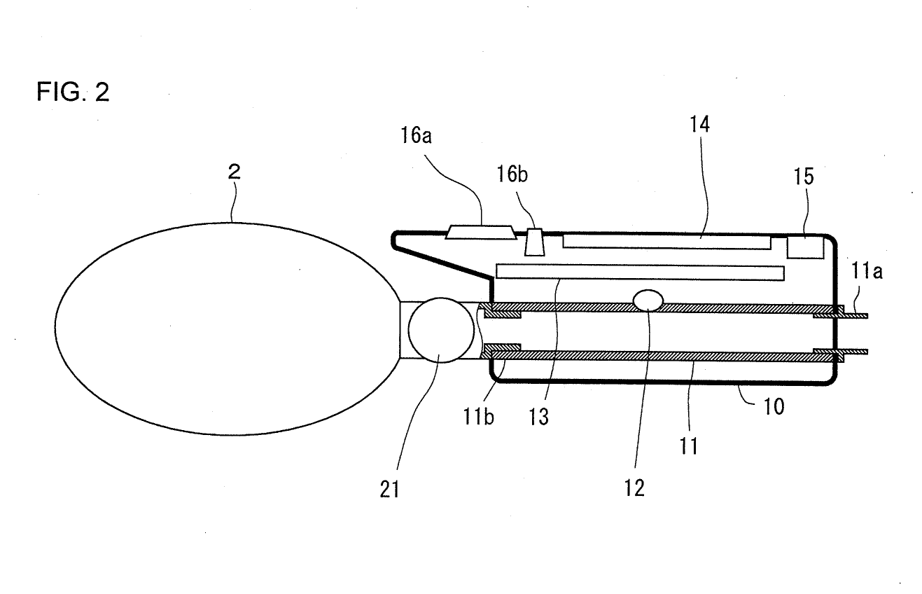

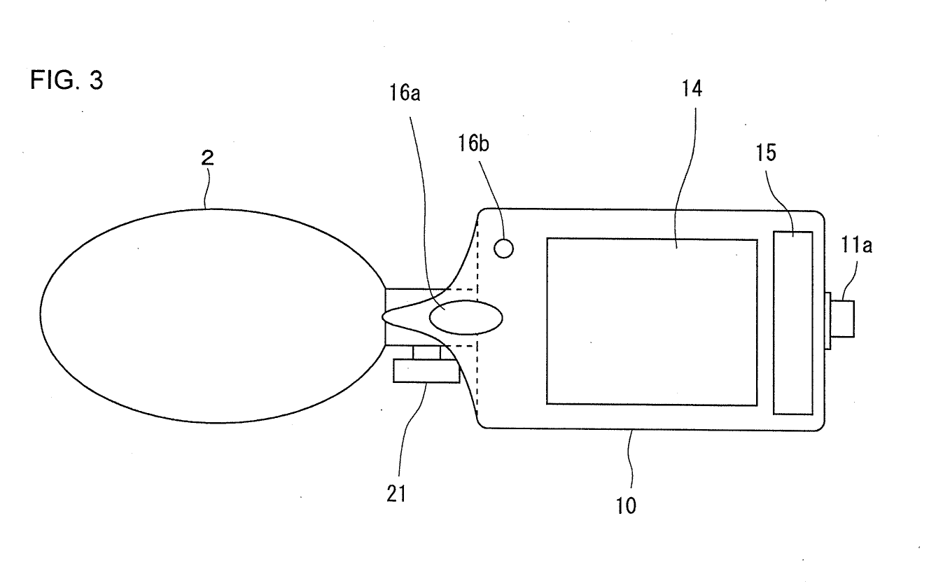

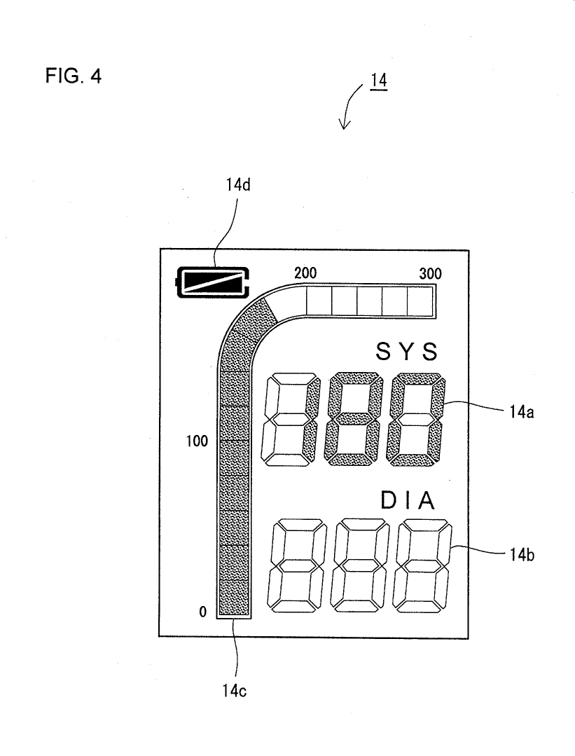

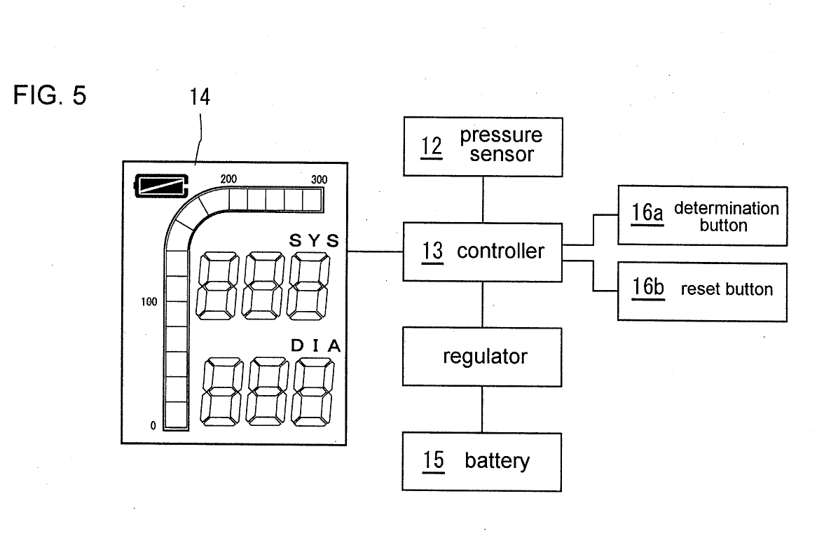

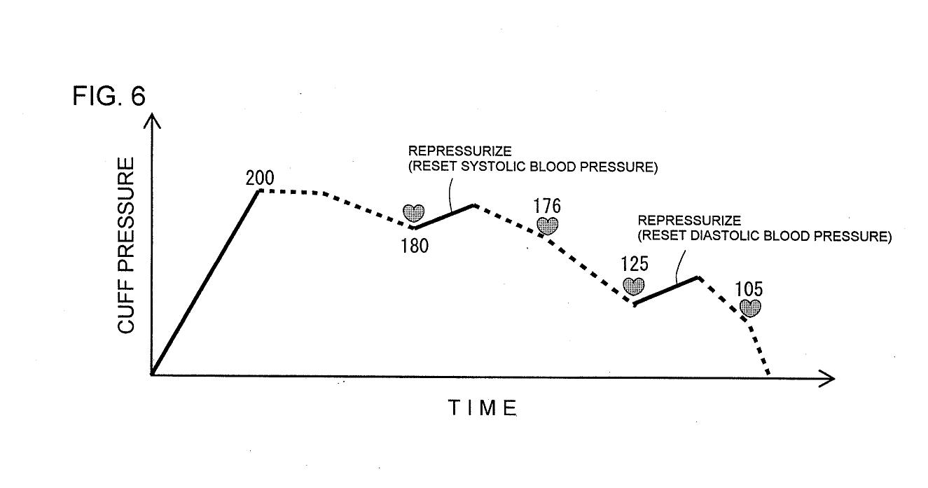

FIGS. 1 to 6 relate to a sphygmomanometer according to an embodiment of the present invention. FIG. 1 is an outline view of the sphygmomanometer, FIG. 2 illustrates a longitudinal sectional view of a main body 1 and a side view of an inflation bulb 2, FIG. 3 is a plan view of the main body 1 and the inflation bulb 2, FIG. 4 is a plan view of a display unit 14, FIG. 5 is a block diagram of the main body 1, and FIG. 6 is a chart that illustrates an example of blood pressure measurement.

The sphygmomanometer according to the embodiment illustrated in FIGS. 1 to 6 includes a main body 1, the inflation bulb 2, a cuff 3, and a tube 4 which connects the main body 1 to the cuff 3. The inflation bulb 2, the cuff 3, and the tube 4 may be similar to those used in a related-art manual sphygmomanometer. The inflation bulb 2 includes a valve 21, through which air is released. The orientation of the valve 21 is made to be changeable by an operator so that the valve 21 can be operated by the operator's dominant hand.

The main body 1 includes a case 10, in which components such as an air duct 11, a pressure sensor 12, a controller 13, the display unit 14, a battery 15, a determination button 16a, and a reset button 16b are incorporated. The air duct 11 is integrally formed with the main body 1 so as to maintain a constant pressure while applying pressure. The determination button 16a is disposed near a valve operating part so as to allow a pressure to be quickly determined.

Although the case 10 uses a plastic box, the material thereof is not limited to plastic. Although the air duct 11 uses a metal having a pipe-like shape, the material thereof is not limited to metal. The pressure sensor 12 may use a commercial pressure sensor. The controller 13 may use a commercial microcomputer (CPU). The display unit 14 may use a liquid crystal display or the like. In the present embodiment, solar cells are used as the battery 15. However, the battery 15 is not limited to this. Alternatively, a battery such as a manganese battery, a lithium-ion battery, or an alkaline battery may be used, or a button cell may be used.

The air duct 11 has an end 11a, to which the tube 4 extending from the cuff 3 is connected, and another end 11b, to which the inflation bulb 2 is connected. Components such as the inflation bulb 2, the valve 21, and the determination button 16a are positioned in such a range that the operator can operate these components with one hand.

The cuff 3, the tube 4, the air duct 11 and the inflation bulb 2 communicate with one another so as to have a substantially uniform inner pressure. The pressure sensor 12, which measures pressure inside the air duct 11, is provided in the air duct 11. The air duct 11 and the cuff 3 communicate with each other and have a uniform inner pressure. Thus, a pressure measured with the pressure sensor 12 will be equal to a cuff pressure.

The tube 4 is easily detachable from the end 11a, and the inflation bulb 2 is also easily detachable from the end 11b. The air compressed and remained in the cuff 3 can be easily completely released by detaching the tube 4 from the end 11a or detaching the inflation bulb 2 from the end 11b.

As illustrated in FIG. 5, in addition to the display unit 14, the pressure sensor 12, the determination button 16a, the reset button 16b, and the battery 15 are connected to the controller 13. The battery 15 is connected to the controller 13 with a regulator connected therebetween. The controller 13 controls contents to be displayed on the display unit 14 responding to output of the pressure sensor 12, the determination button 16a, the reset button 16b, and the battery 15 in accordance with a preset program.

The controller 13 has a function of compensating for measurement errors accompanied by changes in the use environment (temperature).

A 0-point setting of the controller 13 is not affected by differences in the use environment (air pressure). 0 mmHg can be set for the sphygmomanometer in any location by performing the 0-point setting in a disconnected individual sphygmomanometer (main body 1 [symbol] [symbol] ?) in a location where the sphygmomanometer is used. The 0-point setting is performed when the sphygmomanometer is newly used, when the battery is replaced, or when there is change in environment. The 0-point once set will be maintained during use. In the case where the sphygmomanometer has been left unoperated for a certain period of time after measurement, the power of the sphygmomanometer is automatically turned off. In order to use again the turned off sphygmomanometer, pressure is applied (air is pumped) or a switch is used to turn the power on. When the turned off turned off sphygmomanometer is used again, a function with which the previously measured values are held or cleared may be added.

As illustrated in FIG. 4, the display unit 14 includes a systolic blood pressure display 14a, a diastolic blood pressure display 14b, a cuff pressure display 14c, and a battery charge display 14d. The systolic blood pressure display 14a displays a measured systolic blood pressure in a digital (number) display. The diastolic blood pressure display 14b displays a measured diastolic blood pressure in a digital (number) display. The cuff pressure display 14c is a bar-like indicator that indicates a real time cuff pressure and realizes the cuff pressure indication similar to that of a related-art mercury manometer. FIG. 4 illustrates a state in which a value of systolic blood pressure, 180, is measured.

The display unit 14 may include a color or monochrome liquid crystal display panel or both. Furthermore, by incorporating a back-light, the display can be seen in a dark room.

Next, control performed by the controller 13 of the sphygmomanometer according to the present embodiment will be specifically described.

In order to measure blood pressure, the cuff 3 is wrapped around an upper arm of a subject, and air is pumped into the cuff 3 using the inflation bulb 2 so as to set a cuff pressure to be greater than an expected systolic blood pressure. Then, the valve 21 of the inflation bulb 2 is loosened so as to gradually decrease the cuff pressure while sounds from the stethoscope are listened for. When Korotkoff sounds are heard, the determination button 16a is pressed. As a result, a systolic blood pressure is continuously displayed in the display unit. Thus, when the determination button 16a is pressed for the first time after the cuff pressure starts to decrease, the controller 13 causes the cuff pressure determined by the determination button 16a to be displayed as the systolic blood pressure. This display of the systolic blood pressure is maintained until the display is reset.

Repressurization using the inflation bulb 2 is allowed when the operator thinks that the determination button 16a was pressed at a wrong timing for measurement of the systolic blood pressure. When repressurization is performed after the systolic blood pressure has been displayed, the controller 13 causes the systolic blood pressure display 14a to be reset and the cuff pressure measured when the determination button 16a is pressed after repressurization to be newly displayed as the systolic blood pressure. Thus, the systolic blood pressure can be measured again.

After measurement of the systolic blood pressure is completed, by further gradually decreasing the cuff pressure and pressing the determination button 16a when Korotkoff sounds become inaudible, the diastolic blood pressure is continuously displayed on the display unit 14. Thus, when the determination button 16a is pressed after the systolic blood pressure has been displayed and repressurization has not been performed, the controller 13 causes the cuff pressure determined by the determination button 16a to be displayed as the (new [symbol] ) diastolic blood pressure. This display of the diastolic pressure is maintained until the display is reset.

Repressurization using the inflation bulb 2 is allowed when the operator thinks that the determination button 16a was pressed at a wrong timing for measurement of the diastolic blood pressure. When repressurization is performed and the cuff pressure is increased up to a value equal to or smaller than a pressure displayed in the systolic blood pressure display 14a, the controller 13 causes the diastolic blood pressure display 14b to be reset and the cuff pressure determined next time by the determination button 16a to be newly displayed as the diastolic blood pressure. Thus, the diastolic blood pressure can be also measured again.

When the cuff pressure is increased up to be greater than the pressure displayed in the systolic blood pressure display 14a due to repressurization, the controller 13 causes both the systolic and diastolic blood pressure displays 14a and 14b to be reset. In this case, measurement will be performed again from measurement of the systolic blood pressure.

When measurement is completed, the display unit 14 displays the systolic blood pressure and the diastolic blood pressure. According to need, the measurement results can be used , for example, by being jotted down the measurement results. When the main body 1 is equipped with a recording medium such as a secure digital (SD) card, measurement results can be accumulated and used in an external personal computer (PC). And when the main body 1 is equipped with a data output terminal, a recording medium such as a printer can be connected to the main body 1 so as to print measurement results, or the measurement results can be output to an external PC. Furthermore, by incorporating a wireless local area network (LAN) unit, the measurement results can be output to an external PC or the like at a desired timing.

When the reset button 16b is pressed after a set of systolic and diastolic blood pressures have been measured, the systolic blood pressure display 14a and the diastolic blood pressure display 14b are reset and the sphygmomanometer becomes ready for the next measurement.

FIG. 6 illustrates an example of measurement performed using the sphygmomanometer according to the present embodiment. The horizontal axis indicates time, and the vertical axis indicates the cuff pressure.

The cuff 3 is wrapped around an upper arm of the subject. Air is pumped into the cuff 3 using the inflation bulb 2 so as to set the cuff pressure to 200. Next, the valve 21 of the inflation bulb 2 is loosened so as to gradually decrease the cuff pressure while sounds from the stethoscope are listened for. When Korotkoff sounds are heard, the determination button 16a is pressed. Thus, a systolic blood pressure of 180 is displayed. However, the operator thinks that the determination button 16a was pressed at a wrong timing and performs repressurization using the inflation bulb 2. This resets the systolic blood pressure display 14a. After that, the valve 21 of the inflation bulb 2 is loosened so as to gradually decrease the cuff pressure while sounds from the stethoscope are listened for. When Korotkoff sounds are heard, the determination button 16a is pressed. Thus, a systolic blood pressure of 176 is displayed. The cuff pressure is further decreased while sounds from the stethoscope are being heard. When Korotkoff sounds become inaudible, the determination button 16a is pressed. Thus, a diastolic blood pressure of 125 is displayed. However, the operator thinks that the determination button 16a was pressed at a wrong timing and performs repressurization using the inflation bulb 2. This resets the diastolic blood pressure display 14b. After that, the valve 21 of the inflation bulb 2 is loosened so as to gradually decrease the cuff pressure while sounds from the stethoscope are being heard. When Korotkoff sounds become inaudible, the determination button 16a is pressed. Thus, a diastolic blood pressure of 105 is displayed. As a result of this, when the measurement is completed, a systolic blood pressure of 176 and a diastolic blood pressure of 105 are displayed.

The sphygmomanometer according to the present invention may use an external pump and an automatic air release valve connected to the sphygmomanometer instead of the inflation bulb 2, so that pressure can be automatically increased and decreased. Furthermore, an automatic sphygmomanometer is obtainable by incorporating an acoustic sensor that detects Korotkoff sounds into the cuff 3 and adding an external automatic intake and exhaust function.

The main body 1 of the sphygmomanometer according to the present invention may have a hole (or a hook) to which a strap can be attached. This can realize a fall-preventive and portable structure. A pocket hook may be provided on the rear surface of the main body 1 so that the sphygmomanometer can be hung while being carried.

The main body 1 may be contained in a shock-resistant cover so as to absorb shocks given when the sphygmomanometer dropped.

Soft materials like silicon are suitable for the shock-resistant cover. The shock-resistant cover may also have a hole (or hook) for a strap and a pocket hook.

1. A sphygmomanometer comprising:
a main body;
a cuff; and
an inflation bulb,
wherein the main body includes
an air duct having an end connected to a tube extending from the cuff and another end connected to the inflation bulb,
a pressure sensor that measures a pressure in the air duct,
a display unit,
a controller that controls display operation of the display unit in accordance with a measurement obtained using the pressure sensor, and
a determination button,
wherein, the cuff, the tube, the air duct and the inflation bulb communicate with one another so as to have a substantially uniform inner pressure, and
wherein the controller causes the display unit to continuously display the pressure in the air duct measured when the determination button is pressed.

2. The sphygmomanometer according to claim 1,
wherein the display unit includes a systolic blood pressure display and a diastolic blood pressure display,
wherein the pressure in the air duct measured when the determination button is pressed for the first time during a process of decreasing the pressure in the air duct is continuously displayed as the systolic blood pressure,
wherein the pressure in the air duct measured when the determination button is pressed for the second time is continuously displayed as the diastolic blood pressure, and
wherein operations of the display unit to display the systolic blood pressure and the diastolic blood pressure are controlled by the controller.

3. The sphygmomanometer according to claim 2,
wherein the controller causes the systolic blood pressure display to be reset when the pressure in the air duct is increased due to repressurization after the systolic blood pressure has been displayed, and
wherein the pressure in the air duct measured when the determination button is pressed after the systolic blood pressure display has been reset is continuously displayed as the systolic blood pressure.

4. The sphygmomanometer according to claim 3,
wherein the controller causes the diastolic blood pressure display to be reset when the pressure in the air duct is increased up to be equal to or smaller than the pressure displayed in the systolic blood pressure display due to repressurization after the diastolic blood pressure has been displayed, and
wherein the pressure in the air duct measured when the determination button is pressed after the diastolic blood pressure display has been reset is continuously displayed as the diastolic blood pressure.

5. The sphygmomanometer according to claim 4,
wherein the controller causes the systolic blood pressure display and the diastolic blood pressure display to be reset when the pressure in the air duct is increased up to be greater than the pressure displayed in the systolic blood pressure display due to repressurization after the diastolic blood pressure has been displayed, and
wherein the pressure in the air duct measured when the determination button is pressed after the systolic blood pressure display and the diastolic blood pressure display have been reset is continuously displayed as the systolic blood pressure.

6. The sphygmomanometer according to claim 1,
wherein the display unit further includes a cuff pressure display, and
wherein the controller causes the cuff pressure display to display the real time pressure in the air duct.

7. The sphygmomanometer according to claim 2,
wherein the display unit further includes a cuff pressure display, and
wherein the controller causes the cuff pressure display to display the real time pressure in the air duct.

8. The sphygmomanometer according to claim 3,
wherein the display unit further includes a cuff pressure display, and
wherein the controller causes the cuff pressure display to display the real time pressure in the air duct.

9. The sphygmomanometer according to claim 4,
wherein the display unit further includes a cuff pressure display, and
wherein the controller causes the cuff pressure display to display the real time pressure in the air duct.

10. The sphygmomanometer according to claim 5,
wherein the display unit further includes a cuff pressure display, and
wherein the controller causes the cuff pressure display to display the real time pressure in the air duct.
